(19) United States
(12) Patent Application Publication
(10) Pub. No.: US 2020/0337745 A1
(43) Pub. Date: Oct. 29, 2020

(54) HALLUX VALGUS TRACTION DEVICE

(71) Applicant: Orthomis Medical, Inc., Taipei City (TW)

(72) Inventors: Meng-Shin YEN, Taipei City (TW); Cheng-Yu LIU, Taipei City (TW); Chung-Ping CHUANG, Taipei City (TW)

(21) Appl. No.: 16/749,458

(22) Filed: Jan. 22, 2020

(30) Foreign Application Priority Data
Apr. 25, 2019 (TW) 108114596

(51) Int. Cl. A61B 17/72

(52) U.S. Cl. CPC A61B 17/7291

(57) ABSTRACT

A hallux valgus traction device is disclosed. The hallux valgus traction device includes a first traction unit, a second traction unit and a connection unit. The first traction unit and the second traction unit wind around a first metatarsal bone and a second metatarsal bone respectively. The first traction unit and the second traction unit are connected to each other through the connection unit. When the connection unit is tightened, the connection unit brings the first traction unit and the second traction unit to approach each other, so that the first metatarsal bone and the second metatarsal bone also approach each other.

### BACKGROUND

### Field

The disclosure relates to hallux valgus; in particular, to a hallux valgus traction device.

### Description of the Related Art

Hallux valgus is a common foot disease, the main cause of which is due to prolonged over-rotation of the foot, resulting in forefoot ligament relaxation and muscle atrophy, making the metatarsus excessive flexion and rotation, leading to lateral arch collapse, which in turn aggravates the discomfort of the foot and leads to hallux eversion.

If the patient wears shoes with a narrow toe or high heels, the metatarsus will be subjected to considerable pressure, and the front end of the foot will be in a tight space, so that the affected portion of the hallux valgus constantly rubs against the inner surface of the toe and aggravates the discomfort of the affected portion.

Although there are many related products for the treatment of hallux valgus, such as insoles, socks, etc., the effect achieved is still quite limited. In addition, if the patient adopts a costly and complicated surgical treatment method, although it can effectively correct the deformation and improve the degree of pain, the operation needs to cut the bone, destroy the original bone structure, and take up to several months to recover.

### SUMMARY

In view of this, this disclosure provides a hallux valgus traction device, which can reduce the angle between the two metatarsals and shorten the distance without breaking the bone structure, so as to effectively solve the symptoms of hallux valgus.

An embodiment of the disclosure is a hallux valgus traction device. In this embodiment, the hallux valgus traction device includes a first traction unit, a second traction unit and a connection unit. The first traction unit and the second traction unit wind around a first metatarsal bone and a second metatarsal bone respectively, and the first traction unit and the second traction unit are connected to each other through the connection unit. When the connection unit is tightened, the connection unit brings the first traction unit and the second traction unit to approach each other, so that the first metatarsal bone and the second metatarsal bone also approach each other. The first traction unit includes a first connection portion, a first line, a first band and a second line. The first connection portion is connected to the second traction unit through the connection unit. The first connection portion is disposed on one side of the first metatarsal bone near the second metatarsal bone. A first end of the first line is connected to a first end of the first connection portion. A first end of the first band is connected to a second end of the first line. A first end of the second line is connected to a second end of the first band and a second end of the second line is connected to a second end of the first connection portion.

In an embodiment, the first connection portion has a plurality of holes for the connection unit, the first end of the first line and the second end of the second line to pass through to be connected to the first connection portion.

In an embodiment, the first connection portion is made of rigid material to provide support strength.

In an embodiment, the first line, the first band and the second line are made of elastic material so as to surround the first metatarsal bone.

Another embodiment of the disclosure is also a hallux valgus traction device. In this embodiment, the hallux valgus traction device includes a first traction unit, a second traction unit and a connection unit. The first traction unit and the second traction unit wind around a first metatarsal bone and a second metatarsal bone respectively, and the first traction unit and the second traction unit are connected to each other through the connection unit. When the connection unit is tightened, the connection unit brings the first traction unit and the second traction unit to approach each other, so that the first metatarsal bone and the second metatarsal bone also approach each other. The second traction unit includes a second connection portion, a third line, a second band and a fourth line. The second connection portion is connected to the first traction unit through the connection unit. The second connection portion is disposed on one side of the second metatarsal bone near the first metatarsal bone. A first end of the third line is connected to a first end of the second connection portion. A first end of the second band is connected to a second end of the third line. A first end of the fourth line is connected to a second end of the second band and a second end of the fourth line is connected to a second end of the second connection portion.

In an embodiment, the second connection portion has a plurality of holes for the connection unit, the first end of the third line and the second end of the fourth line to pass through to be connected to the second connection portion.

In an embodiment, the second connection portion is made of rigid material to provide support strength.

In an embodiment, the third line, the second band and fourth line are made of elastic material so as to surround the second metatarsal bone.

In an embodiment, when the connection unit is tied with a knot to be fixed, the first traction unit and the second traction unit are maintained a state approaching each other, and the first metatarsal bone and the second metatarsal bone are also maintained a state approaching each other.

Compared to the prior art, the effect of the hallux valgus traction device of the disclosure for correcting hallux valgus symptoms is significantly better than that of the related products such as insoles, socks and the like on the market, and only needs to be performed through minimally invasive surgery, which effectively avoids conventional surgical treatment defects of bone structure and long recovery time.

The advantage and spirit of the disclosure may be understood by the following detailed descriptions together with the appended drawings.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a schematic diagram showing a hallux valgus traction device of an embodiment of the disclosure.

FIG. 2 is a detailed schematic diagram of the first connection portion and the second connection portion in the hallux valgus traction device of the disclosure.

FIG. 3 is a schematic diagram showing that the first traction unit and the second traction unit in the hallux valgus traction device surround the first metatarsal bone and the second metatarsal bone respectively in the disclosure.

FIG. 4 is a schematic diagram showing that the first traction unit and the second traction unit are adjusted respectively, so that the first connection portion is disposed on one side of the first metatarsal bone near the second metatarsal bone and the second connection portion is disposed on one side of the second metatarsal bone near the first metatarsal bone.

FIG. 5 is a schematic diagram of an embodiment of the hallux valgus traction device in the disclosure.

FIG. 6 is a schematic diagram of an embodiment of the hallux valgus traction device being applied to the first metatarsal bone and the second metatarsal bone.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Reference will now be made in detail to the exemplary embodiments of the present disclosure, and examples of the exemplary embodiments will be described in the accompanying drawings. The same or similarly labeled elements/components are used in the drawings and embodiments to represent the same or similar parts.

An embodiment of the disclosure is a hallux valgus traction device. In this embodiment, the hallux valgus traction device can be disposed on the patient's thumb and forefoot through minimally invasive surgery to tract the first metatarsal bone B1 and the second metatarsal bone B2 to approach each other, so that the hallux valgus symptoms can be significantly improved.

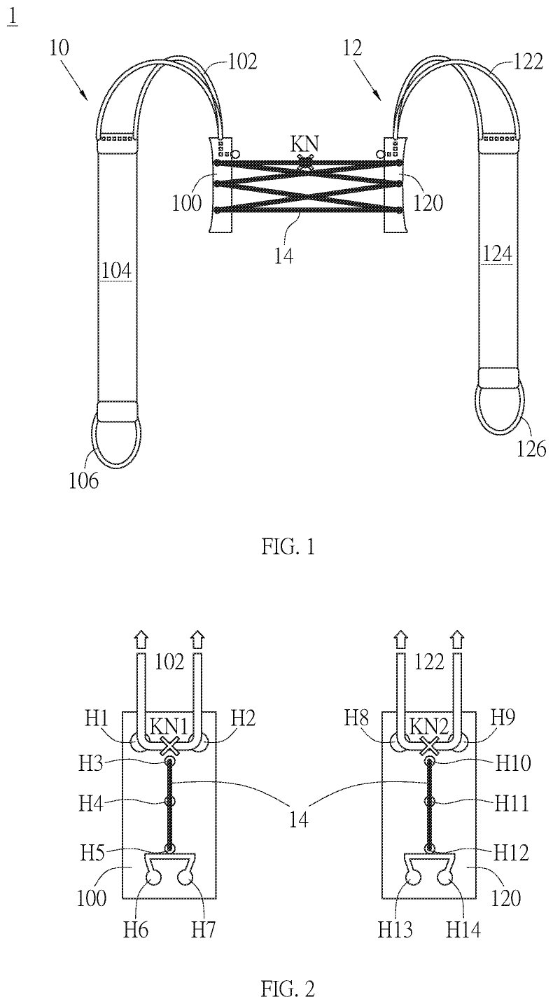

Please refer to FIG. 1. FIG. 1 is a schematic diagram showing the hallux valgus traction device in this embodiment. As shown in FIG. 1, the hallux valgus traction device 1 includes a first traction unit 10, a second traction unit 12 and a connection unit 14. The first traction unit 10 and the second traction unit 12 are connected to each other through the connection unit 14.

The first traction unit 10 includes a first connection portion 100, a first line 102, a first band 104 and a second line 106. The second traction unit 12 includes a second connection portion 120, a third line 122, a second band 124 and a fourth line 126. The first connection portion 100 of the first traction unit 10 and the second connection portion 120 of the second traction unit 12 are connected to each other through the connection unit 14.

A first end of the first line 102 is connected to a first end of the first connection portion 100. A first end of the first band 104 is connected to a second end of the first line 102. A first end of the second line 106 is connected to a second end of the first band 104 and a second end of the second line 106 is connected to a second end of the first connection portion 100.

A first end of the third line 122 is connected to a first end of the second connection portion 120. A first end of the second band 124 is connected to a second end of the third line 122. A first end of the fourth line 126 is connected to a second end of the second band 124 and a second end of the fourth line 126 is connected to a second end of the second connection portion 120.

In practical applications, the first connection portion 100 and the second connection portion 120 can have a rigid material to provide greater support strength, but not limited to this. The first line 102, the first band 104, the second line 106, the third line 122, the second band 124 and the fourth line 126 can have an elastic material so as to surround the bone, but not limited to this.

In addition, the connection unit 14 can be wound around the first connection portion 100 and the second connection portion 120 respectively in a manner similar to tie shoe laces and then tied with a knot KN to be fixed, so that the first connection portion 100 and the second connection portion 120 can be firmly connected to each other by the connection unit 14.

Next, please refer to FIG. 2. FIG. 2 is a detailed schematic diagram of the first connection portion 100 and the second connection portion 120 in the hallux valgus traction device 1. As shown in FIG. 2, the first connection portion 100 has a plurality of holes H1˜H7 and the second connection portion 120 has a plurality of holes H8˜H14.

In this embodiment, the plurality of holes H1˜H7 of the first connection portion 100 are used for the connection unit 14, the first end of the first line 102 and the second end of the second line 106 to pass through to be connected to the first connection portion 100 respectively.

In detail, the holes H1˜H2 are used for the first line 102 to pass through, so that the first line 102 can be connected to the first connection portion 100, and the first line 102 passing through the holes H1˜H2 can be tied by the knot KN1 to be fixed, so that the first line 102 can be firmly connected to the first connection portion 100. The holes H3˜H5 are used for the connection unit 14 to pass through, so that the connection unit 14 can be connected to the first connection portion 100. The holes H6˜H7 are connected to each other, so that the second line 106 can pass through and hook the holes H6˜H7 to be connected to the first connection portion 100.

Similarly, the plurality of holes H8˜H14 of the second connection portion 120 are used for passage of the first end of the third line 122, the connection unit 14, and the second end of the fourth line 126 to connect to the second connection portion 120.

In detail, the holes H8˜H9 are used for the third line 122 to pass through, so that the third line 122 can be connected to the second connection portion 120. The third line 122 passing through the holes H8˜H9 can be tied by the knot KN2 to be fixed, so that the third line 122 can be firmly connected to the second connection portion 120. The holes H10˜H12 are used for the connection unit 14 to pass through, so that the connection unit 14 can be connected to the second connection portion 120. The hole H13˜H14 are connected to each other, so that the fourth line 126 can pass through and hook the holes H13˜H14 to be connected to the second connection portion 120.

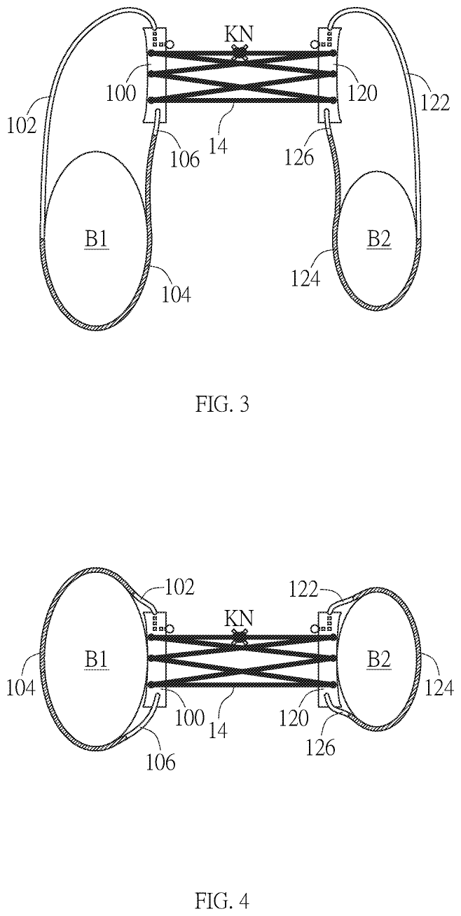

Next, please refer to FIG. 3. FIG. 3 is a schematic diagram showing the first traction unit 10 and the second traction unit 12 in the hallux valgus traction device 1 surrounding the first metatarsal bone B1 and the second metatarsal bone B2 respectively. In practical applications, the first metatarsal bone B1 or the second metatarsal bone B2 can be a toe, but not limited to this.

As shown in FIG. 3, the first traction unit 10 surrounds the first metatarsal bone B1 using the first line 102, the first band 104 and the second line 106. The second traction unit 12 surrounds the second metatarsal bone B2 using the third line 122, the second band 124 and the fourth line 126, but not limited to this.

In detail, the process of the first traction unit 10 surrounding the first metatarsal bone B1 can include the following steps 1A-1D:

Step 1A: the first end and the second end of the first band 104 are connected to the second end of the first line 102 and the first end of the second line 106 respectively;

Step 1B: the first end of the first line 102 is connected to the first end of the first connection portion 100;

Step 1C: the first band 104 surrounds the first metatarsal bone B1; and

Step 1D: the second end of the second line 106 is connected to the second end of the first connection portion 100.

Similarly, the process of the second traction unit 12 surrounding the second metatarsal bone B2 can include the following steps 2A-2D:

Step 2A: the first end and the second end of the second band 124 are connected to the second end of the third line 122 and the first end of the fourth line 126 respectively;

Step 2B: the first end of the third line 122 is connected to the first end of the second connection portion 120;

Step 2C: the second band 124 surrounds the second metatarsal bone B2; and

Step 2D: the second end of the fourth line 126 is connected to the second end of the second connection portion 120.

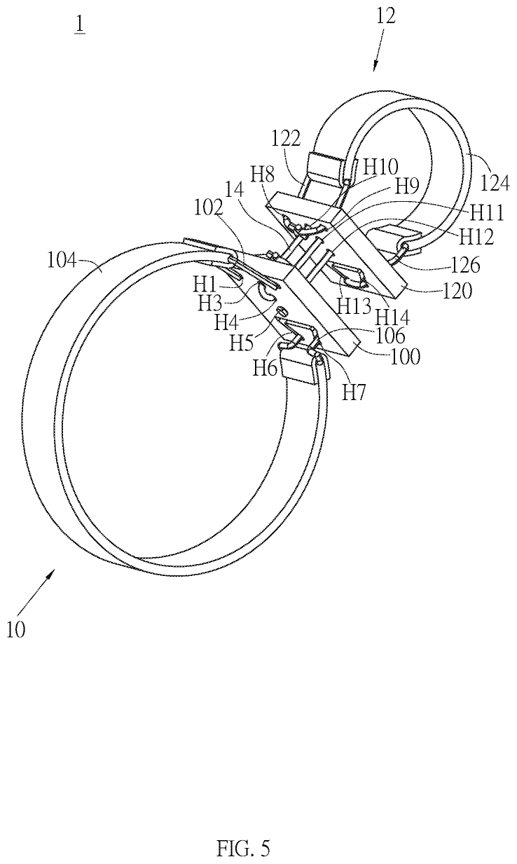

Next, as shown in FIG. 4, when the first traction unit 10 surrounds the first metatarsal bone B1, the first traction unit 10 can be appropriately adjusted so that the first connection portion 100 can be disposed on one side of the first metatarsal bone B1 near the second metatarsal bone B2, but not limited to this.

Similarly, when the second traction unit 12 surrounds the second metatarsal bone B2, the second traction unit 12 can also be appropriately adjusted, so that the second connection portion 120 can be disposed on one side of the second metatarsal bone B2 near the first metatarsal bone B1, but not limited to this.

In this way, the first connection portion 100 disposed on one side of the first metatarsal bone B1 near the second metatarsal bone B2 and the second connection portion 120 on one side of the second metatarsal bone B2 near the first metatarsal bone B1 will be opposed to each other and connected to each other through the connection unit 14.

When the connection unit 14 is tightened, the connection unit 14 will bring the first connection portion 100 of the first traction unit 10 and the second connection portion 120 of the second traction unit 12 to approach each other. At this time, the first band 104 of the first traction unit 10 surrounding the first metatarsal bone B1 will bring the first metatarsal bone B1 to approach the second metatarsal bone B2, and the second band 124 of the second traction unit 12 surrounding the second metatarsal bone B2 will also bring the second metatarsal bone B2 to approach the first metatarsal bone B1, so that the first metatarsal bone B1 and the second metatarsal bone B2 can approach each other.

Since the connection unit 14 connecting the first connection portion 100 of the first traction unit 10 and the second connection portion 120 of the second traction unit 12 can be tied with a knot KN to be fixed, the first connection portion 100 of the first traction unit 10 and the second connection portion 120 of the second traction unit 12 can be continuously maintained in a state approaching each other, so that the first metatarsal bone B1 and the second metatarsal bone B2 can also be continuously maintained in a state approaching each other, thereby the hallux valgus can be effectively improved.

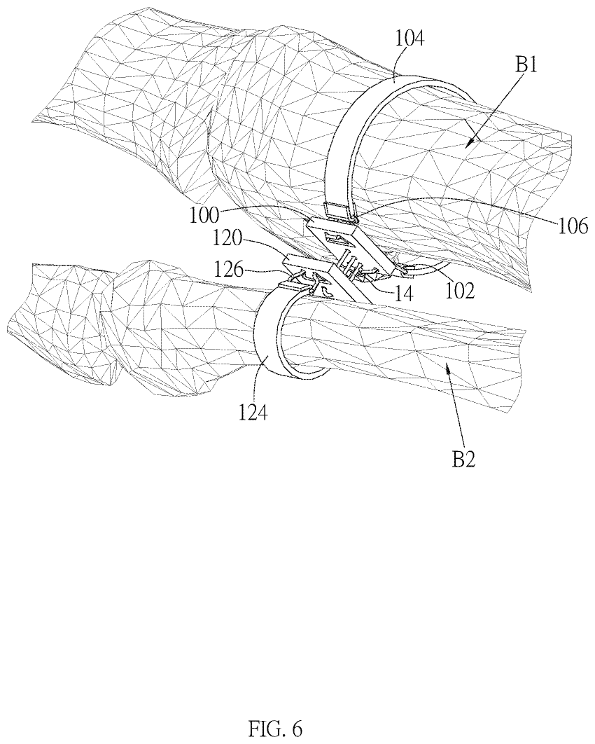

Next, please refer to FIG. 5 and FIG. 6. FIG. 5 illustrates an embodiment of the hallux valgus traction device 1 of the disclosure. FIG. 6 illustrates an embodiment of the hallux valgus traction device 1 applied to the first metatarsal bone B1 and the second metatarsal bone B2. As for its detailed operations, please refer to the foregoing description, which will not be repeated here.

Compared to the prior art, the effect of the hallux valgus traction device of the disclosure for correcting hallux valgus symptoms is significantly better than that of the related products such as insoles, socks and the like on the market, and only needs to be performed through minimally invasive surgery, which effectively avoids conventional surgical treatment defects of bone structure and long recovery time.

With the example and explanations above, the features and spirits of the disclosure will be hopefully well described. Those skilled in the art will readily observe that numerous modifications and alterations of the device may be made while retaining the teaching of the disclosure. Accordingly, the above disclosure should be construed as limited only by the metes and bounds of the appended claims.

What is claimed is:

1. A hallux valgus traction device, comprising:
a first traction unit, configured to surround a first metatarsal bone;
a second traction unit, configured to surround a second metatarsal bone; and
a connection unit, configured to connect the first traction unit and the second traction unit;
wherein when the connection unit is tightened, the connection unit brings the first traction unit and the second traction unit to approach each other, so that the first metatarsal bone and the second metatarsal bone approach each other, and
wherein the first traction unit comprises:
a first connection portion, connected to the second traction unit through the connection unit and disposed on one side of the first metatarsal bone near the second metatarsal bone;
a first line, wherein a first end of the first line is connected to a first end of the first connection portion;
a first band, wherein a first end of the first band is connected to a second end of the first line; and
a second line, wherein a first end of the second line is connected to a second end of the first band, and a second end of the second line is connected to a second end of the first connection portion.

2. The hallux valgus traction device of claim 1, wherein the first connection portion has a plurality of holes for the connection unit, the first end of the first line and the second end of the second line to pass through to be connected to the first connection portion.

3. The hallux valgus traction device of claim 1, wherein the first connection portion is made of rigid material to provide support strength.

4. The hallux valgus traction device of claim 1, wherein the first line, the first band and the second line are made of elastic material so as to surround the first metatarsal bone.

5. A hallux valgus traction device, comprising:
a first traction unit, configured to wind around a first metatarsal bone;
a second traction unit, configured to wind around a second metatarsal bone; and
a connection unit, configured to connect the first traction unit and the second traction unit;
wherein when the connection unit is tightened, the connection unit brings the first traction unit and the second traction unit to approach each other, so that the first metatarsal bone and the second metatarsal bone approach each other, and
wherein the second traction unit comprises:
a second connection portion, connected to the first traction unit through the connection unit and disposed on one side of the second metatarsal bone near the first metatarsal bone;
a third line, wherein a first end of the third line is connected to a first end of the second connection portion;
a second band, wherein a first end of the second band is connected to a second end of the third line; and
a fourth line, wherein a first end of the fourth line is connected to a second end of the second band and a second end of the fourth line is connected to a second end of the second connection portion.

6. The hallux valgus traction device of claim 5, wherein the second connection portion has a plurality of holes for the connection unit, the first end of the third line and the second end of the fourth line to pass through to be connected to the second connection portion.

7. The hallux valgus traction device of claim 5, wherein the second connection portion is made of rigid material to provide support strength.

8. The hallux valgus traction device of claim 5, wherein the third line, the second band and the fourth line are made of elastic material so as to surround the second metatarsal bone.

9. The hallux valgus traction device of claim 5, wherein when the connection unit is tied with a knot to be fixed, the first traction unit and the second traction unit are maintained a state approaching each other, and the first metatarsal bone and the second metatarsal bone are also maintained the state approaching each other.
